(19) United States
(12) Patent Application Publication
(10) Pub. No.: US 2024/0024569 A1
(43) Pub. Date: Jan. 25, 2024

(54) DEVICE FOR ADMINISTERING AN INFUSION OR TRANSFUSION, SYSTEM COMPRISING SUCH A DEVICE, AND METHOD FOR CONTROLLING SUCH A DEVICE

(71) Applicant: B. Braun Melsungen AG, Melsungen (DE)

(72) Inventor: Andreas Katerkamp, Melsungen (DE)

(21) Appl. No.: 18/025,281

(22) PCT Filed: Sep. 13, 2021
(86) PCT No.: PCT/EP2021/075081

§ 371 Date: Mar. 8, 2023

(30) Foreign Application Priority Data
Sep. 15, 2020 (DE) 10 2020 211 555.9

(51) Int. Cl. A61M 5/142; A61M 5/168; A61M 5/14; A61M 5/165

(52) U.S. Cl. CPC A61M 5/14228; A61M 5/16859; A61M 5/16827; A61M 5/1409; A61M 5/1411; A61M 5/165; A61M 5/16881; A61M 2005/1401; A61M 5/1413; A61M 5/1414; A61M 2005/1657; A61M 2205/106; A61M 2205/126; A61M 2205/3337; A61M 2205/3334; A61M 2202/0007; A61M 2202/0028; A61M 2202/04; A61M 2206/20

(57) ABSTRACT

A device for administering an infusion or transfusion of liquid from a container includes a pump for pumping liquid to a patient connection via a conduit having a liquid-retaining filter membrane. The device also includes a pressure measuring device for acquiring measurement values corresponding to a pump-inlet-side pressure, and a control device for detecting a change in the pump-inlet-side pressure based on the measurement values. The control device determines, based on the change in the pump-inlet-side pressure, whether to stop pumping. The control device also generates a control signal when the pumping is to be stopped. Moreover, the control device emits, based on the control signal, a communication signal to a data connection between the device and a unit that reads the communication signal. In addition, or in the alternative, the control device switches the device from a first state to a second state based on the control signal.

### CROSS-REFERENCE TO RELATED APPLICATIONS

This application is the United States national stage entry of International Application No. PCT/EP2021/075081, filed Sep. 13, 2021, and claims priority to German Application No. 10 2020 211 555.9, filed Sep. 15, 2020. The contents of International Application No. PCT/EP2021/075081 and German Application No. 10 2020 211 555.9 are incorporated by reference herein in their entireties.

### FIELD

The present disclosure is directed to a device for administering an infusion or transfusion, a system comprising an infusion or transfusion device, and a method for controlling an infusion or transfusion device.

### BACKGROUND

Infusions and transfusions are performed for therapeutic purposes in human and veterinary medicine. Infusions and transfusions are used to administer liquids to a patient. For example, infusions may be used to administer liquid drugs (drug solutions, etc.).

An infusion set or transfusion set is understood to be a product with which the administration of a medical infusion or the execution of a medical transfusion or the execution of a comparable administration of a liquid may be carried out. For example, the terms “infusion set”, “infusion or transfusion kit”, or “infusion kit” are also commonly used for an infusion set or transfusion set, wherein the use of the terms “infusion set” and “infusion kit” are not intended to exclude the possibility that the product so designated may also be used, for example, to perform a transfusion.

An infusion set or transfusion set usually comprises a tube and often a drip chamber. The infusion set or transfusion set may optionally include further components such as a flow controller for controlling the flow rate of the liquid, e.g. a roller clamp. The liquid to be administered in the course of an infusion or transfusion is provided in a container. The container may be, for example, an infusion bottle, an infusion bag, a blood bag, etc.

If a drip chamber is provided, it is typically connected to the container via a container connector so that the liquid may exit the container and enter the drip chamber. In the interior of the drip chamber, a drop former is provided which causes the liquid to enter the drip chamber from the container in the form of droplets of normalized size. The container connector may, for example, be a piercing device such as a hollow mandrel that may be used to pierce a septum closing the container and that typically comprises a plurality of channels in its interior. Such a piercing device is commonly referred to as a “spike”. Other systems are also known for connecting the drip chamber to the container, such as coupling systems that do not allow the drip chamber and container to be separated once they have been connected. The drip chamber is in fluid communication with one end of the tube such that liquid may enter the tube from the drip chamber. If a drip chamber is not used, the tube is connected directly to the container or a suitable container connector. The tube comprises a connection for a patient access port (e.g., venous cannula or venous catheter) at the other end. The connection for the patient access port is referred to below as the “patient connection”. The patient access port may optionally also be considered to be an element of the infusion set or transfusion set.

A drip chamber, as described, provides the connection between the tube and the container. Frequently, devices that ensure the ventilation of the system are integrated into the drip chamber. For this purpose, the drip chamber comprises, for example, a vent device with a manually operated or an automatic vent valve and a vent channel open to the interior of the drip chamber. In the prior art, different embodiments of the vent device based on different types of valves and with or without a vent filter are known, for example, manual vent devices that have a manually operated flap as a vent valve and automatic vent valves that comprise a check valve (non-return valve) as a vent valve. Alternatively, the manual or automatic vent device is not integrated into a drip chamber but is located at another suitable position of the fluid system. The present invention is compatible with manual and automatic vent devices. In particular, if the container in which the liquid to be administered is presented is collapsible, the use of a drip chamber or vent device may also be omitted because it is then not necessary to allow air to flow into the system for pressure equalization.

In addition to the gravity infusion or gravity transfusion technique, in which the liquid is delivered from the container to the patient access port solely by the action of gravity, the pump infusion or pump transfusion technique has become established. In pump infusion or pump transfusion, the liquid is conveyed using a pump. By using a pump, the administration of the liquid may be controlled in a better way. For example, the pump may be a peristaltic pump, which engages and periodically deforms a portion of the tube to create a peristaltic pumping motion. Such peristaltic pumps are advantageous because no components of the pump come into contact with the liquid such that there is no risk of contamination caused by the use of the pump. Furthermore, peristaltic pumps are easy to handle. In particular, it is easy to connect a tube to the pump and to disconnect this connection after the infusion is complete. For this purpose, peristaltic infusion or transfusion pumps usually comprise a housing with an open channel or slot into which the tube is inserted.

In the course of administering an infusion or transfusion, it is necessary to prevent large amounts of air from entering the patient's body. Air entering the bloodstream, for example, may cause a life-threatening air embolism. To prevent air from entering the patient's body, for example, it is necessary to fill the tube with the liquid before beginning administration of the liquid to the patient. This preparatory step is often referred to as “priming” or “priming step”.

To prevent air from entering the patient's body, it is also necessary to ensure that the infusion or transfusion is stopped as soon as the liquid to be administered is used up. To facilitate the termination of the infusion or transfusion at the correct time, some of the infusion sets or transfusion sets on the market have a liquid-retaining filter membrane. This liquid-retaining filter membrane is located in the fluid channel through which the liquid passes from the container to the patient access port. For example, the liquid-retaining filter membrane may be located at the bottom of the drip chamber, i.e., near its exit. However, it may also be located elsewhere in the tube. When the liquid is used up to the point that there is essentially no liquid left in the portion of the fluid system between the liquid-retaining filter membrane and the container, a resistance to further flow of the liquid builds up at the liquid-retaining membrane. That is, the liquid-retaining membrane functions as a membrane that resists the flow of the column of liquid located in the tube below the liquid-retaining membrane. The function of the liquid-retaining membrane is described in detail below. In the course of a gravity infusion or transfusion, this effect may be used to stop the flow of liquid as soon as the container or drip chamber is empty. In the course of a pump infusion or pump transfusion, this effect may be exploited in a similar way by configuring the pump such that an alarm signal is sent and/or the pumping of liquid is stopped as soon as the container or drip chamber is empty. Usually, the pumps used have a pressure measuring device that may detect the pressure of the liquid in the pump-inlet-side portion (pump upstream portion) of the tube. The pump-inlet-side portion of the tube is understood to be the portion of the tube located upstream of the pump. The liquid-retaining filter membrane is preferably located upstream of the pump. Therefore, in this case, the portion of the tube upstream of the pump is understood to be the portion between the pump and the liquid-retaining filter membrane. When the container or drip chamber is empty, the pressure of the liquid in the pump-inlet-side portion of the tube decreases. A control device that receives signals from the pressure measuring device is configured to send an acoustic and/or visual alarm signal and/or to switch off the pump when it is determined that the infusion or transfusion has ended.

When an alarm signal has been sent and/or the pump has been switched off, it is necessary for medical staff to take further steps. The further steps may, for example, consist of connecting another infusion or transfusion. The further steps may also consist of, for example, documenting the completion of the infusion or transfusion and disconnecting the infusion set or transfusion set from the patient. In any case, it is necessary that medical staff is on site to perceive the alarm signal and to take the further steps.

### SUMMARY

Based on the above situation, an object of the invention is to provide an improved device for administering an infusion or transfusion, an improved system comprising such a device, and an improved method for controlling such a device.

The device according to the invention is a device for administering an infusion or transfusion of a liquid from a container. The device according to the invention comprises a pump for pumping the liquid to a patient connection via a conduit comprising a liquid-retaining filter membrane. The device according to the invention further comprises a pressure measuring device for acquiring measurement values corresponding to a pump-inlet-side pressure being present inside the conduit between the pump and the liquid-retaining filter membrane. The device according to the invention further comprises a control device. The control device is configured to detect a change in the pump-inlet-side pressure based on the measurement values. The control device is further configured to determine, on the basis of the change in the pump-inlet-side pressure, whether the pumping of the liquid is to be stopped. The control device is further configured to generate a control signal when the pumping of the liquid is to be stopped. The control device is further configured to emit, based on the control signal, a communication signal to a data connection between the device and a unit configured to read the communication signal and/or to switch, based on the control signal, the device from a first active operating state to a second active operating state.

Herein, the data connection may be wireless, e.g., using WIFI.

Herein, the unit configured to read the communication signal may be an external unit, i.e. a unit external to the device according to the invention. This unit may also be an internal unit, for example a display and/or an acoustic signal generator arranged at the pump, in particular in the same housing as the pump. The unit configured to read the communication signal may also be another pump or a switchable multi-way valve.

Herein, an “active operating state” is an operating state in which the device is pumping a liquid. That is, the inactive state of the device, in which no pump is performing any activity, is not an active operating state.

In this context, it is preferred that in the first operating state, the liquid is pumped as a first liquid from the container as a first container to the patient connection, and in the second operating state, another liquid is pumped as a second liquid from another container as a second container to the patient connection. In this case, the control device is configured to cause the device to deliver a further liquid from a further container to the patient connection based on the control signal.

In an embodiment of the invention, the control device is configured to emit, based on the control signal, a communication signal to a data connection between the device and a unit configured to read the communication signal. This unit is preferably an external unit, such that the communication signal may cause a unit external to the device according to the invention to perform an activity. The data connection may be wireless, e.g. by means of WIFI.

This makes it possible, for example, to process the completion of an infusion or transfusion via a computer-based information system. Herein, the computer-based information system may serve various purposes. For example, the computer-based information system may serve to automatically protocol the termination of the infusion and/or record the termination of the infusion in the patient's electronic medical record. For example, the computer-based information system may also serve to notify the responsible medical staff that an infusion has been completed without the need for the medical staff to be on site. The information may be transmitted to a mobile terminal (cell phone, etc.), for example.

By emitting the communication signal to such a unit, it is further possible, for example, to control other medical devices. For example, a medical analyzer may be caused to perform a medical examination on the patient that is required immediately or a predetermined time after administration of the infusion. In this case, it is not required that the medical examination is initiated by the medical staff in a separate step.

This unit may also be another infusion or transfusion administration device that begins administration when it receives an appropriate communication signal. In this way, administration of multiple infusions or transfusions is possible without the need for medical staff to take action between each infusion or transfusion.

This unit may be an external unit, i.e., a unit external to the device according to the invention. This unit may also be an internal unit, for example a display and/or an acoustic signal generator located at the pump, in particular in the same housing as the pump. This unit may also be another pump or a switchable multi-way valve.

In another embodiment of the invention, the control device is configured to cause, based on the control signal, the device to deliver another liquid from another container to the patient connection. This makes it possible, for example, to automatically administer a further liquid from another container after the administration of the liquid from one container has been completed without the need for medical staff to take action. In this way, sequential infusion of liquids from two or more than two containers may be performed in an automated and therefore time and cost saving manner.

In further embodiments of the invention, the above embodiments are combined. For example, the control device may be configured to cause the device to first administer a liquid from one container, then, based on the control signal, cause a medical analyzer to perform a medical examination on the patient to determine whether to administer another liquid from another container, and, if necessary, cause the same device or another device to deliver another liquid from another container to the patient connection.

In all embodiments, the advantage arises from the fact that no specific sensors, other than the pressure measuring device, are required to enable the determination of whether to stop the administration of a liquid. By eliminating the need for additional specific sensors, the complexity of the device and its use as well as the cost of its manufacture and maintenance may be reduced. Eliminating the need for additional specific sensors may also reduce the possibility of failure of the device.

The system according to the invention is a system comprising a device according to the invention and at least one infusion set or transfusion set. The at least one infusion set or transfusion set comprises a conduit with a liquid-retaining filter membrane and a patient connection. The system according to the invention provides the equipment by which a liquid may be conveyed from a container to a patient access port, wherein the beneficial effects of the invention may be achieved.

Preferably, the conduit comprises a drip chamber in which the liquid-retaining filter membrane is arranged in the exit region.

Preferably, the pressure measuring device intermittently acquires the measurement values corresponding to the pump-inlet-side pressure, and further preferably there are equal time intervals between the acquisition of two successive measurement values.

Preferably, the pump is a peristaltic pump.

The method according to the invention is a method for controlling a device for administering an infusion or transfusion of a liquid from a container,

wherein the device comprises:
a pump for pumping the liquid to a patient connection via a conduit comprising a liquid-retaining filter membrane,
a pressure measuring device for acquiring measurement values corresponding to a pump-inlet-side pressure being present inside the conduit between the pump and the drip chamber; and
a control device.

The control device is configured to detect a change in the pump-inlet-side pressure based on the measurement values. The control device is further configured to determine, on the basis of the pump-inlet-side pressure, whether to stop pumping. The control device is further configured to generate a control signal when the pumping of the liquid is to be stopped. The control device is further configured to emit, based on the control signal, a communication signal to a data connection between the device and a unit configured to read the communication signal and/or to switch, based on the control signal, the device from a first active operating state to a second active operating state, for example, by causing the device to deliver another liquid from another container to the patient connection. The data connection may be wireless, for example using WIFI.

By using the method according to the invention, it is in particular possible to control the device according to the invention to achieve the advantageous effects of the invention.

### BRIEF DESCRIPTION OF THE DRAWINGS

Further features, expediencies, and advantages of the invention are described below by way of exemplary embodiments and with reference to the attached figures.

FIG. 1 shows a device according to a first embodiment of the invention as well as a system consisting of the device according to the first embodiment, an infusion set or transfusion set, and a unit configured to read the communication signal, and also shows a container.

FIG. 2 shows a device according to a second embodiment of the invention as well as a system consisting of the device according to the second embodiment and an infusion set or transfusion set, and also shows two containers.

FIG. 3 shows the device according to the second embodiment of the invention as well as a system consisting of the device according to the second embodiment and two infusion sets or transfusion sets, and also shows two containers.

FIG. 4 shows a device according to a third embodiment of the invention as well as a system consisting of the device according to the third embodiment and an infusion set or transfusion set, and also shows two containers.

FIG. 5 shows a device according to a fourth embodiment of the invention.

### DETAILED DESCRIPTION

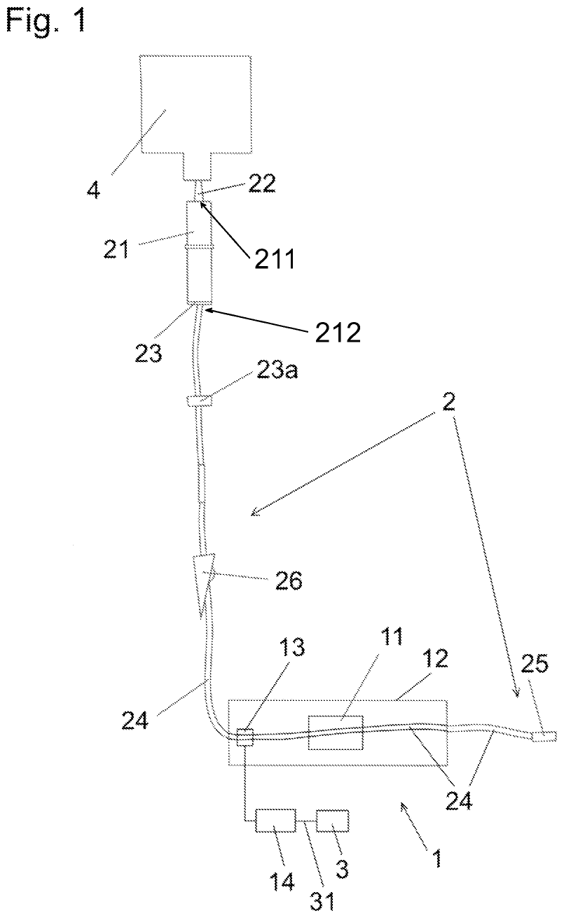

FIG. 1 shows a device 1 according to a first embodiment of the invention and an infusion set or transfusion set 2, which is not part of the device according to the invention. The device 1 and the infusion set or transfusion set 2 together constitute an embodiment of the system according to the invention. Furthermore, a unit 3 configured to read in the communication signal is shown in FIG. 1. Herein, the unit 3 is shown as an external unit 3 that is not part of the system according to the invention. Furthermore, a container 4 in which a liquid to be administered to a patient is provided is shown in FIG. 1. The container 4 is not part of the system according to the invention. The container 4 may, for example, be an infusion bottle, an infusion bag, a blood container, etc.

The infusion set or transfusion set 2 shown specifically in FIG. 1 comprises a drip chamber 21 having an inlet 211 through which the liquid may enter the drip chamber 21, and an outlet 212 through which the liquid may enter the tube leading to the patient. In the operating position of the drip chamber 21, the inlet 211 is at the top while the outlet 212 is at the bottom.

In the portion of the inlet 211, the drip chamber 21 has a container connector 22 for connection to a container 4 such that the liquid may pass from the container 4 into the drip chamber 21. A drop former (not shown in FIG. 1) is provided inside the drip chamber 21 in the portion of the inlet 211, which causes the liquid to enter in the form of drops of normalized size. The container connector 22 shown in FIG. 1 is a piercing device that may be used to pierce a septum closing the container 4, wherein the piercing device typically comprises a plurality of channels in its interior. Such a piercing device is commonly referred to as a “spike”. Alternatively, the container connector may be configured, for example, in the form of another coupling system.

The drip chamber 21 preferably has a vent device not shown in FIG. 1.

A liquid-retaining filter membrane 23 is arranged in the portion of the outlet 212 of the drip chamber. The liquid-retaining filter membrane 23 has the function of preventing the passage of fluid when there is no liquid or only a low liquid level above the membrane. In this way, air is prevented from passing through the membrane such that it is ensured that air does not enter the patient's bloodstream.

This is achieved, for example, in that the membrane has a porous structure and the liquid passing through the membrane flows through the pores or through the channels formed by the pores. According to one theory, the function of such a membrane is explained as follows, although the present invention is not limited to the liquid-retaining membrane functioning according to this theory: According to the theory, if there is at least some amount of liquid above the membrane or liquid is dropped onto the membrane from above, the weight of that liquid provides sufficient pressure to allow the liquid to flow through the pores or channels. If there is no liquid or only a low level of liquid above the membrane, the liquid will be held in the pores or channels as a result of capillary forces and air may be prevented from flowing through the pores or channels. The situation may be imagined in such a way that the liquid in the pores or channels configures a meniscus and does not flow further. This capillary effect is also referred to as “capillary stop flow”. Preferably, the membrane comprises a hydrophilic material because the capillary effect is then more pronounced than in the case of a non-hydrophilic material.

The pressure difference between the top and the bottom of the membrane, beyond which air may enter the membrane to displace the liquid held in the pores or channels by the capillary effect described, is called the breakdown pressure. The term “bubble point pressure” is also used for the breakdown pressure. In operation, there is a column of liquid in the tube below the membrane, which creates, as a result of gravity, a negative pressure on the membrane depending on the length of the liquid column. The breakdown pressure must exceed the negative pressure created by the liquid column to prevent the liquid column from flowing off and air from entering the tube. For gravity infusion, the dimensions of the membrane and its materials and structure are selected and adapted to the infusion liquid and the length of the tube such that the breakthrough pressure is sufficient and the membrane may prevent the liquid column from flowing off. For example, the breakthrough pressure for some commercially available infusion sets with a tube length of approximately 150 cm is at least 20 kPa. 20 kPa corresponds to a water column of 200 cm. Since the tube length and thus the liquid column are shorter, gravity infusion automatically stops the flow as soon as the container or drip chamber is empty. For example, when using an infusion pump, the pump is automatically turned off when the pressure difference between the top and bottom of the membrane comprises a predetermined value that is less than the breakthrough pressure. For example, the pump may be set to shut off at a pressure drop of 17 kPa (corresponding to a pressure in the tube of −17 kPa compared to the pressure that would otherwise be present during the infusion).

The infusion set or transfusion set 2 further comprises a tube 24. In the specific embodiment shown in FIG. 1, the tube 24 is connected to the outlet 212 of the drip chamber 21. In alternative embodiments in which the infusion set or transfusion set 2 does not have a drip chamber, the tube 24 is connected directly to the container connector 22 or directly to the container 4.

Alternatively, the liquid-retaining filter membrane 23 is not arranged in the portion of the outlet of a drip chamber, but in a separate element 23a along the tube 24. In any case, the liquid must pass through the liquid-retaining filter membrane 23, 23a in order to flow from the container to the patient. Further, it is possible that multiple liquid-retaining filter membranes 23, 23a are arranged along the tube 24.

The infusion set or transfusion set 2 further comprises a patient connection 25 connected to one end of the tube 24. The patient connection 25 provides a connection for a patient access port. The patient access port is not shown in FIG. 1. The patient access port may be, for example, a venous cannula, venous catheter, etc. The patient access port may optionally be considered part of the infusion set or transfusion set.

If a drip chamber is not used, the tube 24 is directly connected to the container 4 or a suitable container connector. The liquid-retaining membrane 23a is then arranged at a suitable location along the tube 24.

The infusion set or transfusion set 2 may optionally include further components, such as a flow regulator 26 for shutting off the tube 24 and/or controlling the flow rate of the liquid. The optional flow regulator 26 is exemplified in FIG. 1 as a roller clamp.

The infusion set or transfusion set 2 provides a connection between a container 4, in which the liquid to be administered is provided, and a patient access port. In operation, i.e. during an infusion or transfusion, liquid passes from the container 4 into the tube 24 and through this tube 24 to the patient connection 25, which is connected to the patient access port. Optionally, a drip chamber 21 is provided which provides a connection between the tube 24 and the container connector 22 which is connected to the container 4. The liquid passes through the liquid-retaining filter membrane 23, 23a as it flows from the container 4 to the patient connection 25. The liquid-retaining filter membrane 23, 23a is preferably located upstream of the pump described below.

The infusion set or transfusion set 2 is therefore an example of a conduit comprising a liquid-retaining filter membrane 23, 23a for delivering a liquid to a patient connection 25 connected to the patient access port.

The device 1 includes a pump 11, and the pump 11 is optionally arranged in a housing 12. The pump 11 is, for example, a peristaltic pump that may be engaged with a portion of the tube 24 to periodically deform the tube 24 to produce a peristaltic pumping motion. The peristaltic pumping motion of the wall of the tube 24 causes liquid to be pumped through the tube toward the patient connection 25.

The device 1 further comprises a pressure measuring device 13 for acquiring measurement values corresponding to a pump-inlet-side pressure being present inside the conduit, i.e., inside the tube 24, between the pump and the liquid-retaining filter membrane.

Preferably, the pressure measuring device 13 and the pump 11 are arranged in the same housing 12. This ensures that the pressure measuring device 13 acquires the pressure values at the correct position along the tube 24. Furthermore, this allows for simplicity of operation for the user because the user only needs to connect the tube 24 to one device whose housing 12 includes the pump 11 and the pressure measuring device 13.

The pressure measuring device 13 may, for example, be a measuring device that determines the size of the cross-section of the elastic tube 24 in the portion upstream of the pump. The pressure measuring device 13 may alternatively be, for example, a measuring device that determines the resistance of the elastic tube 24 to a compressive force in the portion upstream of the pump. From the cross-section and the resistance to deformation, conclusions may be drawn about the pressure being present inside the tube 24. Further alternatively, measuring sensors are possible which project into the interior of the tube 24 and determine the pressure directly in contact with the liquid.

The device 1 further comprises a control device 14. In the specific embodiment shown in FIG. 1, the control device 14 is spatially separated from the housing 12 in which the pump 11 is also housed. Alternatively, the control device 14 may also be arranged within the housing 12 of the pump. The control device 14 may include, for example, one or more processors, one or more memories, electrical connections, connectors, interfaces, and other electronic and electrical components. The control device 14 is configured to detect a change in the pump-inlet-side pressure based on the measurement values. The control device 14 is further configured to determine whether to stop pumping the liquid based on the change in the pump-inlet-side pressure. The control device 14 is further configured to generate a control signal when the pumping of the liquid is to be stopped. The control device 14 is further configured to emit, based on the control signal, a communication signal to a data connection 31 between the device 1 and a unit 3 configured to read the communication signal. The data connection 31 may be wireless, for example by means of WIFI.

In operation, i.e. after priming the infusion set or transfusion set 2 and during administration of an infusion or transfusion, the liquid entering the tube 24 from the container 4, optionally via a drip chamber 21, is conveyed by the pump 11 through the tube 24 to the patient, i.e. to the patient connection 25, which is connected to the patient access port. The liquid enters the patient's body, for example a vein, through the patient access port. The pressure measuring device 13 acquires measurement values in this process. The measurement values are a measure of the pump-inlet-side pressure being present inside the conduit, i.e., inside the tube 24, between the pump 11 and the liquid-retaining filter membrane 23, 23a. The pressure measuring device may acquire the measurement values continuously or intermittently.

When the liquid to be administered has been completely withdrawn from the container 4, the pump 11 initially still pumps liquid through the tube 24. As this happens, the liquid level in the drip chamber 21 or in the tube 24 continues to drop. This continues until the liquid level reaches the liquid-retaining filter membrane 23, 23a. When the liquid level reaches the liquid-retaining filter membrane 23, 23a, the pressure of the liquid in the pump-inlet-side portion of the tube between the liquid-retaining filter membrane 23, 23a and the pump 11 decreases because the pump 11 continues to pump from this portion. The decrease in the pump-inlet-side pressure is detected by the control device 14 based on the measurement values acquired by the pressure measuring device 13. The control device 14 therefore detects a change in the pump-inlet-side pressure, in this case in the form of a drop. Based on the change in the pump-inlet-side pressure, the control device further determines whether the pumping of the liquid is to be stopped. This is the case when the control device determines that a criterion related to the change in pressure or to the measurement values is fulfilled. The criterion may be, for example: Falling below a predetermined threshold value of the pressure; falling below a predetermined threshold value of the difference quotient of the pressure calculated on the basis of time increments; falling below a predetermined threshold value of the differential quotient corresponding to the time derivative of the pressure.

When the control device 14 has determined that the pumping of the liquid is to be stopped, it generates a corresponding control signal.

When the control device 14 has generated a corresponding control signal, it emits a corresponding communication signal to a data connection 31.

The control signal is therefore a signal that is processed within the control device 14, causing a communication signal to be emitted. In other words, the control signal is a command that causes the part of the control device 14 whose function is to emit signals to emit the communication signal by emitting it to the data connection 31.

The data connection 31 is not part of the system according to the invention. The data connection 31 is for connecting the control device 14 and the unit 3 configured to read the communication signal. The data connection 31 may, for example, be a cable, an optical data conductor, a wireless connection, etc. In particular, the data connection 31 may be a wireless connection using WIFI. The unit 3 is configured to read the communication signal. That is, the control device 14 is vice versa configured to emit a communication signal that may be read in by the unit 3.

The unit 3 may be, for example, a preferably electronic control device, a computer, a computer network, a medical analyzer, etc. The communication signal, for example, causes the unit 3 to store data in the patient's electronic medical record or in a directory in which infusions or transfusions are documented, or to initiate a further action desired following the infusion or transfusion. The further action may be, for example, the administration of a further infusion or transfusion by another device or the execution of a medical examination.

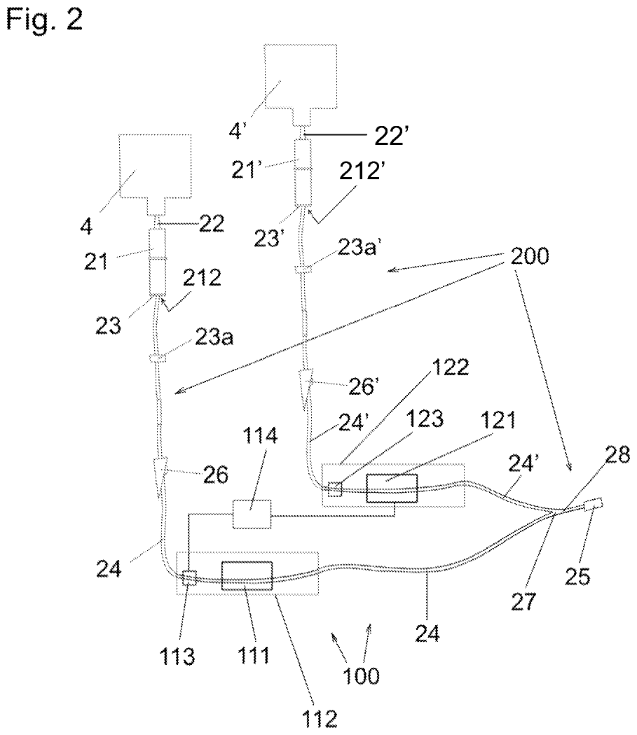

FIG. 2 shows a device 100 according to a second embodiment of the invention and an infusion set or transfusion set 200, which is not part of the device according to the invention. The device 100 and the infusion set or transfusion set 200 together represent an embodiment of the system according to the invention.

Insofar as elements of the infusion set or transfusion set 200 shown specifically in FIG. 2 are analogous in structure and function to the elements described in connection with the first embodiment with reference to FIG. 1, they are designated below with reference signs corresponding to the above reference signs. Where necessary or appropriate, an apostrophe is used below to assign the reference signs to each of these elements when more than one of them is shown in FIG. 2.

In addition to the device 100 and the infusion or transfusion set 200, two containers 4 and 4′, each of which may provide a liquid to be administered to a patient, are shown in FIG. 2. The containers 4 and 4′ are not part of the system according to the invention. The containers 4 and 4′ may be, for example, infusion bottles, infusion bags, blood containers, etc.

The infusion set or transfusion set 200 shown specifically in FIG. 2 has two tubes 24 and 24′ that join at a tube connection point 27 to form a common section of tubing 28. The tubes 24 and 24′ are two supply lines that lead in the direction of flow to the tube connection point 27. The common tube section 28 is a discharge line leading away from the tube connection point. The common tube section 28 is connected to a patient connection 25. In all other respects, the tubes 24 and 24′ correspond to the tube 24 described above in connection with the first embodiment. The patient connection 25 provides a connection for a patient access port. The patient access port is not shown in FIG. 2.

The presence of a drip chamber 21 or 21′ is optional for each of the tubes 24 or 24′, respectively. With regard to the details of the drip chamber, reference is made to the above description for the first embodiment.

In the region of the outlet 212, 212′ of each of the drip chambers 21, 21′ shown in FIG. 2, a liquid-retaining filter membrane 23, 23′ is arranged. With regard to the details of the liquid-retaining filter membrane, reference is made to the above description for the first embodiment.

Alternatively, the liquid-retaining filter membrane 23, 23′ is not arranged in the region of the outlet of a drip chamber, but in a separate element 23a, 23a′ along the tube 24, 24′. In any case, the liquid must pass through the liquid-retaining filter membrane 23, 23′, 23a, 23a′ in order to flow from the container to the patient. Furthermore, it is possible that several liquid-retaining filter membranes are arranged along the tube 24, 24′.

The infusion set or transfusion set 200 may optionally include further components such as one or more flow regulators 26, 26′ for shutting off and/or controlling the flow rate of the liquid. The optional flow regulators 26, 26′ are shown as exemplary roller clamps in FIG. 2.

The infusion set or transfusion set 200 provides a connection between the containers 4 and 4′, each of which provides a liquid to be administered, and a patient access port. In operation, that is, during an infusion or transfusion, a liquid is delivered from one of the containers 4 and 4′ to the patient connection 25, which is connected to the patient access port. The liquid passes through the respective liquid-retaining filter membrane 23, 23′, 23a, 23a′ on its way from the respective container 4, 4′ to the patient port 25. The liquid-retaining filter membranes 23, 23′, 23a, 23a′ are preferably arranged upstream of the pumps described below.

Thus, the infusion set or transfusion set 200 is an example of a branched conduit that includes a liquid-retaining filter membrane and that is used to deliver liquids to a patient access port connected to the patient connection 25.

The device 100 includes two pumps 111 and 121, each optionally arranged within a housing 112, 122. The pumps 111 and 121 are, for example, peristaltic pumps. With regard to the details concerning peristaltic pumps, reference is made to the above description concerning the first embodiment.

The device 100 further comprises a pressure measuring device 113 for determining measurement values corresponding to a pump-inlet-side pressure being present inside the conduit, i.e. inside the tube 24, between the pump 111 and the respective liquid-retaining filter membrane 23, 23′, 23a, 23a′. In this regard, the device 100 preferably comprises two pressure measuring devices 113 and 123, each for acquiring measurement values corresponding to a pump-inlet-side pressure being present inside the conduit, i.e. inside the tube 24 or 24′, between the respective pump 111, 121 and the respective liquid-retaining filter membrane 23, 23′, 23a, 23a′. With regard to the details concerning the pressure measuring devices 113 and 123, reference is made to the above description concerning the first embodiment.

Preferably, the pressure measuring device 113 and the pump 111 are arranged in a common housing 112 and the pressure measuring device 123 and the pump 121 are arranged in a common housing 122.

The device 100 further comprises a control device 114. In the specific embodiment shown in FIG. 2, the control device 114 is spatially separated from the housings 112, 122 of the pumps 111, 121. Alternatively, the control device 114 may be located in one of the housings 112 and 122. Further alternatively, both pumps 111 and 121 are arranged in a common housing, in which the control device 114 may also be arranged. The control device 114 is arranged to detect a change in the pressure on the pump-inlet-side based on the measurement values acquired by the pressure measuring device 113. The control device 114 is further arranged to determine, based on the change in the pump-inlet-side pressure whether the pumping of the liquid from the first container 4 is to be stopped. The control device 114 is further adapted to generate a control signal when the pumping of the liquid is to be stopped. Based on the control signal, the control device is further adapted to cause the device 100 to deliver a further liquid from a further container 4′ to the patient, i.e., to the patient port 25.

In operation, i.e., after priming the infusion set or transfusion set 200 and during administration of an infusion or transfusion, the liquid entering the tube 24 from the first container 4, optionally via a drip chamber 21, is pumped by the pump 111 through the tube 24 to the patient, i.e., to the patient connection 25, which is connected to the patient access port. The liquid enters the patient's body, for example a vein, through the patient access port. During this process, the pressure measuring device 113 acquires measurement values. The measurement values are a measure of the pump-inlet-side pressure that is present inside the conduit, i.e., inside the tube 24, between the pump 111 and the liquid-retaining filter membrane 23, 23a. The pressure measuring device 113 may acquire the measurement values continuously or intermittently.

When the liquid to be administered has been completely withdrawn from the first container 4, the pump 11 initially still delivers liquid through the tube 24. As this happens, the liquid level in the drip chamber 21 or in the tube 24 continues to drop. This continues until the liquid level reaches the liquid-retaining filter membrane 23, 23a. When the liquid level reaches the liquid-retaining filter membrane 23, 23a, the pressure of the liquid in the pump-inlet-side portion of the tube between the liquid-retaining filter membrane 23, 23a and the pump 111 decreases as the pump 111 continues to pump from this portion. The decrease in the pump-inlet-side pressure is detected by the control device 114 based on the measurement values acquired by the pressure measuring device 113. The control device 114 therefore detects a change in the pump-inlet-side pressure in the tube 24, in this case in the form of a drop in pressure. Based on the change in the pump-inlet-side pressure, the control device 114 further determines whether the delivery of the liquid from the first container 4 is to be stopped. This is the case when the control device determines that a criterion related to the change in pressure or to the measurement values is met. The criterion may be, for example: Falling below a predetermined threshold value of the pressure; falling below a predetermined threshold value of the difference quotient of the pressure calculated on the basis of time increments; falling below a predetermined threshold value of the differential quotient corresponding to the time derivative of the pressure.

When the control device 114 has determined that the pumping of the liquid from the first container is to be stopped, it generates a corresponding control signal.

This control signal causes the device 100 to deliver another liquid from a second container 4′ to the patient connection.

In this way, the infusion or transfusion of the liquid from the first container 4 is automatically stopped and the infusion or transfusion of the liquid from the second container 4′ is automatically started, which greatly reduces the time required by medical staff when administering multiple infusions or transfusions in succession to the same patient, i.e., administering a sequential infusion or transfusion.

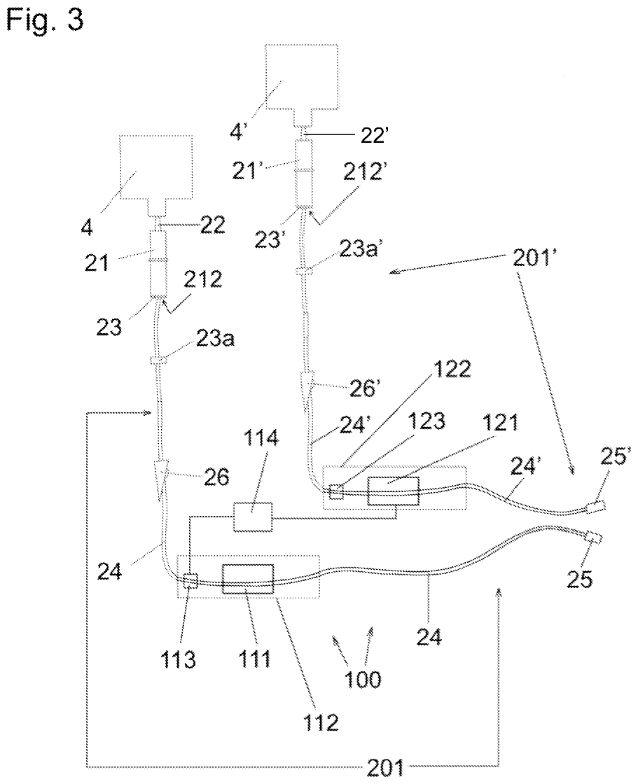

FIG. 3 shows the device 100 according to the second embodiment of the invention and two infusion sets or transfusion sets 201 and 201′, which are not part of the device according to the invention. The device 100 and the infusion set or transfusion set 200 together represent a further embodiment of the system according to the invention.

The configuration shown in FIG. 3 corresponds to the configuration shown in FIG. 2, except that instead of one infusion set or transfusion set with two tubes joining at a tube junction, there are two separate infusion sets or transfusion sets 201 and 201′. In other words, in the situation shown in FIG. 2, an infusion set or transfusion set 200 with a branch is associated with both pumps 111 and 121, while in the situation shown in FIG. 3, a separate infusion set or transfusion set 201, 201′ is associated with each pump 111 and 121.

The infusion sets or transfusion sets 201 and 201′ each comprise their own patient connection 25 and 25′, respectively. The patient connections 25 and 25′ each serve to connect to a patient access port. In all other respects, reference is made to the above description of the system illustrated in FIG. 2.

The device according to the second embodiment has two pumps 111 and 121, each for conveying the liquid from one of the containers 4, 4′ in the context of an automatic sequential infusion or transfusion.

The same concept is applied to automatic sequential infusion or transfusion of liquids from more than two containers in further embodiments. For this purpose, the device according to the invention has a corresponding number of pumps. Together with the device, an infusion set or transfusion set with a corresponding number of tubes is used. Alternatively, a corresponding number of infusion sets or transfusion sets may be used. The device according to the invention and the infusion set or transfusion set with a corresponding number of tubes or the device according to the invention and a corresponding number of infusion sets or transfusion sets then each form systems according to the invention.

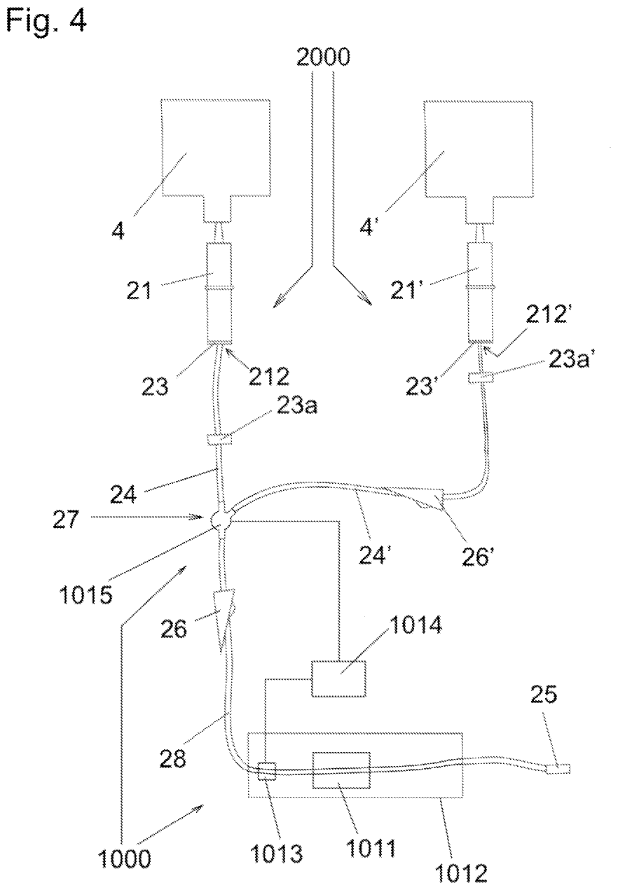

FIG. 4 shows a device 1000 according to a third embodiment of the invention as well as an infusion set or transfusion set 2000 that is not part of the device according to the invention. The device 1000 and the infusion set or transfusion set 2000 together constitute an embodiment of the system according to the invention.

Insofar as elements of the infusion set or transfusion set 2000 shown specifically in FIG. 4 are analogous in structure and function to the elements described in connection with the first embodiment with reference to FIG. 1, they are designated below with reference signs corresponding to the above reference signs. Where necessary or appropriate, an apostrophe is used below to assign the reference signs to each of these elements when more than one of them is shown in FIG. 4.

In addition to the device 1000 and the infusion set or transfusion set 2000, two containers 4 and 4′, each of which may provide a liquid to be administered to a patient, are shown in FIG. 4. The containers 4 and 4′ are not part of the system according to the invention. The containers 4 and 4′ may, for example, be infusion bottles, infusion bags, blood containers, etc.

The infusion set or transfusion set 2000 shown specifically in FIG. 4 comprises two tubes 24 and 24′, which join at a tube connection point 27 through a switchable multi-way valve 1015 to form a common tube section 28. The tubes 24 and 24′ are two supply lines leading in the direction of flow to the tube connection point 27. The common tube section 28 is a discharge line leading away from the tube connection point 27. The common tube section 28 is connected to a patient connection 25. In all other respects, the tubes 24 and 24′ correspond to the tube 24 described above in connection with the first embodiment. The patient connection 25 provides a connection for a patient access port. The patient access port is not shown in FIG. 4. The multi-way valve 1015 may be, for example, a magnetic multi-way valve and/or an actuator-operated multi-way valve.

The provision of a drip chamber 21 or 21′ is optional for each of the tubes 24 or 24′, respectively. With regard to the details concerning the drip chamber, reference is made to the above description concerning the first embodiment.

A liquid-retaining filter membrane 23, 23′ is arranged in the portion of the outlet 212, 212′ of each of the drip chambers 21, 21′ shown in FIG. 4. With regard to the details of the liquid-retaining filter membrane, reference is made to the above description for the first embodiment.

Alternatively, the liquid-retaining filter membrane 23a, 23a′ is not arranged in the portion of the outlet of a drip chamber, but in a separate element 23a, 23a′ along the tube 24, 24′. In any case, the liquid must pass through the liquid-retaining filter membrane 23, 23′, 23a, 23a′ in order to flow from the container to the patient. Further, it is possible that multiple liquid-retaining filter membranes 23, 23′, 23a, 23a′ are arranged along the tube 24, 24′.

The infusion set or transfusion set 2000 may optionally include other components, such as one or more flow regulators 26, 26′ for shutting off and/or controlling the flow rate of the liquid. The optional flow regulators 26, 26′ are shown as exemplary roller clamps in FIG. 4.

The infusion set or transfusion set 2000 provides a connection between the containers 4 and 4′, each of which provides a liquid to be administered, and a patient access port. In operation, i.e., during an infusion or transfusion, liquid is conveyed from one of the containers 4 and 4′ to the patient, i.e., to the patient connection 25 which is connected to the patient access port. The liquid passes through the respective liquid-retaining filter membrane 23, 23, 23a, 23a′ on its way from the respective container 4, 4′ to the patient connection 25. The liquid-retaining filter membranes 23, 23′, 23a, 23a′ are preferably arranged upstream of the pump described below.

Thus, the infusion set or transfusion set 2000 is an example of a conduit comprising a liquid-retaining filter membrane for delivering liquids to a patient access port connected to the patient connection 25.

The device 1000 includes a pump 1011, and the pump 1011 is optionally arranged within a housing 1012. The pump 1011 is, for example, a peristaltic pump. With regard to details concerning peristaltic pumps, reference is made to the above description concerning the first embodiment.

The device 1000 further comprises a pressure measuring device 1013 for acquiring measurement values corresponding to a pump-inlet-side pressure being present inside the conduit, i.e. inside the tube section 28 and the tube 24 or the tube 24′ between the pump 1011 and the respective liquid-retaining filter membrane 23, 23′, 23a, 23a′.

Preferably, the pressure measuring device 1013 and the pump 1011 are arranged in the same housing 1012.

The device 1000 further comprises a control device 1014. In the specific embodiment shown in FIG. 4, the control device 1014 is spatially separated from the housing 1012 of the pump 1011. Alternatively, the control device 1014 may be arranged in the housing 1012. The control device 1014 is configured to detect a change in the pump-inlet-side pressure based on the measurement values acquired by the pressure measuring device 1013. The control device 1014 is further configured to determine, based on the change in the pump-inlet-side pressure, whether the pumping of the liquid from the first container 4 is to be stopped. The control device 1014 is further configured to generate a control signal when the pumping of the liquid is to be stopped. The control device is further configured to cause, based on the control signal, the device 1000 to deliver another liquid from another container 4′ to the patient connection 25. For this purpose, the device 1000 comprises a multi-way valve 1015. The multi-way valve 1015 is switchable from a first position to a second position based on the control signal. In the first position, the path from the tube 24 into the tube 28 is open and the path from the tube 24′ into the tube 28 is closed. In the second position, the path from the tube 24 into the tube section 28 is closed and the path from the tube 24′ into the tube section 28 is open. When the multi-way valve 1015 receives the control signal, it automatically switches from the first position to the second position.

In operation, i.e., after priming the infusion set or transfusion set 2000 and during administration of an infusion or transfusion, the liquid entering the tube 24 from the first container 4, optionally via a drip chamber 21, is first pumped by the pump 1011 through the tube 24, the multi-way valve 1015, and the tube section 28 to the patient connection 25, which is connected to the patient access port. The liquid enters the patient's body, such as a vein, through the patient access port. Herein, the pressure measuring device 1013 acquires measurement values. The measurement values are a measure of the pump-inlet-side pressure being present inside the conduit, i.e., inside the tube 24 and the tube section 28 between the pump 1011 and the liquid-retaining filter membrane 23. The pressure measuring device 113 may acquire the measurement values continuously or intermittently.

When the liquid to be administered has been completely withdrawn from the first container 4, the pump 1011 initially still pumps liquid through the tube 24. As this happens, the liquid level in the drip chamber 21 or in the tube 24 continues to drop. This continues until the liquid level reaches the liquid-retaining filter membrane 23, 23a. When the liquid level reaches the liquid-retaining filter membrane 23, 23a, the pressure of the liquid in the pump-inlet-side portion of the tube between the liquid-retaining filter membrane 23, 23a and the pump 1011 decreases as the pump 1011 continues to pump from this portion. The decrease in the pump-inlet-side pressure is detected by the control device 1014 based on the measurement values acquired by the pressure measuring device 1013. Therefore, the control device 1014 detects a change in the pump-inlet-side pressure. Based on the change in the pump-inlet-side pressure, the control device 1014 further determines whether to stop pumping the liquid from the first container 4. This is the case when the control device determines that a criterion related to the change in pressure or to the measurement values is met. The criterion may be, for example: Falling below a predetermined threshold value of the pressure; falling below a predetermined threshold value of the difference quotient of the pressure calculated on the basis of time increments; falling below a predetermined threshold value of the difference quotient corresponding to the time derivative of the pressure.

When the control device 1014 has determined that the delivery of the liquid from the first container 4 has to be stopped, it generates a corresponding control signal.

This control signal causes the device 1000 to switch the multi-way valve 1015 from the first position to the second position to deliver another liquid from a second container 4′ to the patient connection. At the same time, the multi-way valve 1015 shuts off the tube 24 such that no air is pumped through the tube 24.

In this way, the infusion or transfusion of the liquid from the first container 4 is automatically stopped and the infusion or transfusion of the liquid from the second container 4′ is automatically started, greatly reducing the time required by medical staff when administering multiple infusions or transfusions sequentially to the same patient, i.e., administering a sequential infusion or transfusion.

The device according to the third embodiment has a multi-way valve 1015 that may be switched between a first position and a second position to allow the pumps 1011 to sequentially deliver liquids from the first container 4 and from the second container 4′ as part of an automated sequential infusion or transfusion to be administered to the patient.

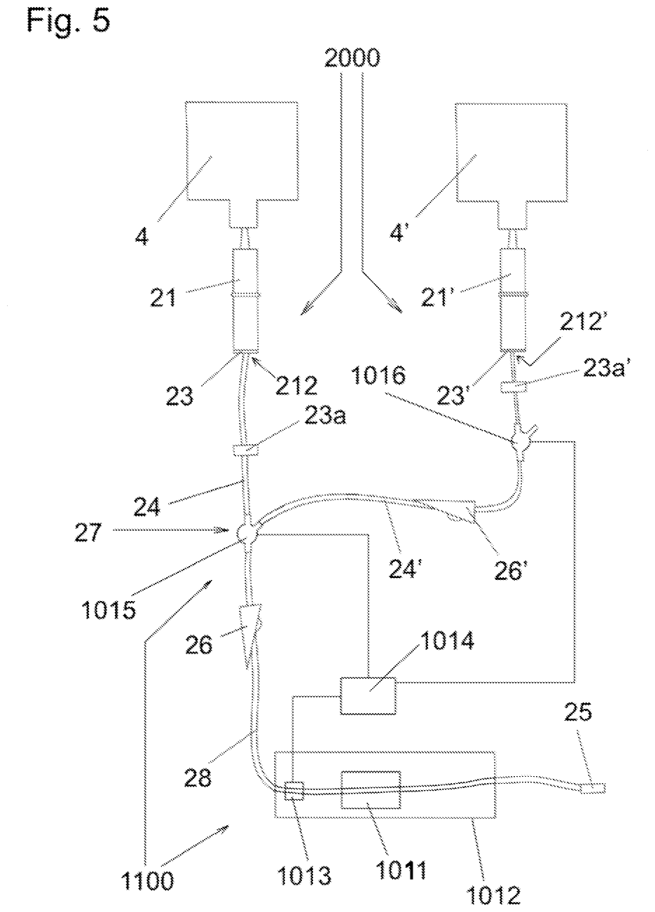

The same concept is applied to automatic sequential infusion or transfusion of liquids from more than two containers in further embodiments. For this purpose, the device according to the invention has a multi-way valve with a corresponding number of paths. Together with the device, an infusion set or transfusion set with a corresponding number of tubes is used. Alternatively, multiple multi-way valves may be provided. FIG. 5 illustrates a fourth embodiment of the device 1100 according to the invention. The device 1100 has a second multi-way valve 1016. Otherwise, the device 1100 according to the fourth embodiment is configured analogously to the device 1000 according to the third embodiment. The second multi-way valve 1016 allows the line to be branched a second time such that another supply line (not shown in FIG. 5) may be connected through which liquid may be delivered from a third container (not shown in FIG. 5) toward the patient. The second multi-way valve 1016 is switchable from a first position to a second position based on a control signal. In the first position, the path for the liquid from the third container is closed. In the second position, the path for the liquid from the third container is open. When the control device 1014 has determined that the pumping of the liquid from the second container 4 is to be stopped, it generates an appropriate control signal. As a result of the control signal, the second multi-way valve 101 switches from the first position to the second position to allow another liquid to be delivered from the third container to the patient connection. At the same time, the second multi-way valve 1016 blocks the path for the liquid from the second container 4′ such that no air is pumped through the line. In this way, automatic sequential infusion or transfusion of liquids from three containers may be realized.

In an analogous manner, automatic sequential infusion or transfusion of liquids from more than three containers may be realized. This requires the provision of more than three multi-way valves or a multi-way valve that may be switched between a sufficient number of positions.

In further embodiments, the control device 14, 114, 1014 of the device 1, 100, 1000, 1100 is configured to check whether the pumping of the liquid is completed and whether a communication signal is to be sent to a data connection 31 between the device and a unit 3 configured to read the communication signal and/or whether the device 100, 1000, 1100 is to be caused to pump a further liquid from a further container 4′ to the patient. In other words, the control device 14, 114, 1014 then checks whether the pumping of the liquid from the current container is completed and whether a further liquid from a further container is to be pumped or a communication signal is to be emitted. For this purpose, the control device 14, 114, 1014 causes, for example, the pump 11, 111, 121, 1011 to temporarily reverse the pumping direction, i.e., to pump in a direction away from the patient. Based on the measurement values acquired by the pressure measuring device 13, 113, 123, 1013, the control device 14, 114, 1014 determines whether there is liquid on the side of the liquid-retaining filter membrane 23, 23′, 23a, 23a′ facing away from the pump 11, 111, 121, 1011. The control device 14, 114, 1014 is further configured to generate the control signal depending on the result of the check. That is, the control device 14, 114, 1014 generates the control signal when the check indicates that the delivery of the liquid from the current container has been completed. For example, a check criterion may be to determine whether the measurement values acquired by the pressure measuring device 13, 113, 123, 1013 when the pumping direction is reversed correspond to a pressure increase in the conduit between the pump 11, 111, 121, 1011 and the liquid-retaining filter membrane 23, 23′, 23a, 23a that is greater than a predetermined threshold value. If this is the case, there is a malfunction of the liquid-retaining filter membrane 23, 23′, 23a, 23a. For example, the liquid-retaining filter membrane 23, 23′, 23a, 23a may be clogged. The control device may respond to this, for example, by causing the device 1, 100, 1000, 1010 to continue the infusion or transfusion from another container or to switch to an alarm state. When the pressure increase is only small and the pressure value stabilizes rapidly, the situation is that the liquid level has reached the liquid-retaining filter membrane 23, 23′, 23a, 23a before reversing the pumping direction, such that the control signal is generated and, based on the control signal, a communication signal is emitted to a data connection 31 between the device 1 and a unit 3, which is configured to read in the communication signal, and/or based on the control signal, the device 100, 1000, 1100 is caused to deliver a further liquid from a further container 4′ to the patient. To prevent liquid from being sucked out of the patient when the pumping direction is reversed, it is advantageous if the infusion set or transfusion set has at least one suitable check valve.

1. A device for administering an infusion or transfusion of a liquid from a container,
wherein the device comprises:
a pump for pumping the liquid to a patient via a conduit comprising a liquid-retaining filter membrane;
a pressure measuring device for acquiring measurement values corresponding to a pump-inlet-side pressure being present inside the conduit between the pump and the liquid-retaining filter membrane; and
a control device,
wherein the control device is configured to detect a change in the pump-inlet-side pressure based on the measurement values,
wherein the control device is further configured to determine, based on the change in the pump-inlet-side pressure, whether pumping of the liquid is to be stopped,
wherein the control device is further configured to generate a control signal when pumping of the liquid is to be stopped, and
wherein the control device is further configured to emit, based on the control signal, a communication signal to a data connection between the device and a unit configured to read the communication signal and/or to switch, based on the control signal, the device from a first active operating state to a second active operating state.

2. The device according to claim 1, wherein in the first active operating state, the liquid is delivered to the patient as a first liquid from the container as a first container and in the second active operating state a further liquid is delivered to the patient as a second liquid from a further container as a second container, and/or
wherein, for switching the device from the first to the second active operating state, a further pump is controlled as a second pump in addition to or instead of the pump as a first pump, and/or
wherein a multi-way valve is controlled for switching the device from the first to the second active operating state.

3. The device according to claim 1, wherein the control device is configured to detect a drop in the pump-inlet-side pressure on the basis of the measurement values and to determine on the basis of the drop whether the pumping of the liquid is to be stopped and the control signal is to be generated, and/or
wherein the control device is configured to determine whether the pumping of the liquid is to be stopped and the control signal is to be generated by comparing the measurement values or a difference quotient calculated on the basis of the measurement values or differential quotient calculated on the basis of the measurement values with a threshold value.

4. The device according to claim 1, wherein the control device is further configured to check whether the pumping of the liquid is completed and whether a communication signal is to be emitted to a data connection between the device and a unit, which is configured to read the communication signal, and/or whether the device is to be caused to deliver a further liquid from a further container to the patient,
wherein the control device is further configured to generate the control signal depending on the result of the checking.

5. The device according to claim 1, wherein the conduit comprises a drip chamber arranged upstream of the pump, wherein the drip chamber comprises an inlet, through which the liquid from the container may enter in the form of drops, and wherein drip chamber comprises an outlet, through which the liquid may flow into the remaining conduit, wherein the liquid-retaining filter membrane is arranged between the inlet and the outlet.

6. The device according to claim 1, wherein the device comprises, in addition to the pump as first pump for pumping the liquid, at least one further pump as a second pump for pumping a further liquid to the patient,
wherein the control device is configured to deactivate the first pump and activate the second pump to cause the further liquid to be delivered to the patient by the second pump.

7. The device according to claim 1, wherein the control device is configured to switch a multi-way valve arranged in the conduit such that subsequently to delivering the liquid as the first liquid, a further liquid as the second liquid is delivered to the patient with the pump.

8. The device according to claim 1, wherein the pressure measuring device and the pump are arranged in the same housing.

9. A system comprising:
a device according to claim 1; and
at least one infusion or transfusion set comprising a conduit with a liquid-retaining filter membrane and a patient connection.

10. The system according to claim 9, wherein the liquid-retaining filter membrane is an air-stop membrane, and/or
wherein the liquid-retaining filter membrane is a hydrophilic and/or porous liquid-retaining membrane.

11. The system according to claim 9, wherein the device comprises at least a first pump and a second pump, wherein the system comprises at least two infusion or transfusion sets, and wherein the first pump and the second pump are each associated with one of the at least two infusion or transfusion sets.

12. The system according to claim 9, wherein the device comprises at least a first pump and a second pump, wherein the system further comprises an infusion or transfusion set associated with the first pump and the second pump.

13. The system according to claim 9, wherein the conduit of the at least one infusion or transfusion set comprises a multi-way valve at which at least a first and a second supply line and a discharge line connected to the patient connection join.

14. A method of controlling a device for administering an infusion or transfusion of a liquid from a container,
wherein the device comprises:
a pump for pumping the liquid to a patient via a conduit comprising a liquid-retaining filter membrane,
a pressure measuring device for acquiring measurement values corresponding to a pump-inlet-side pressure being present inside the conduit between the pump and the liquid-retaining filter membrane, and
a control device,
wherein the control device is configured to determine a change in the pump inlet side pressure on the basis of the measurement values,
wherein the control device is further configured to determine, on the basis of the change in the pump-inlet-side pressure, whether the pumping of the liquid is to be stopped,
wherein the control device is further configured to generate a control signal when the pumping of the liquid is to be stopped, and
wherein the control device is further configured to emit, based on the control signal, a communication signal to a data connection between the device and a unit configured to read the communication signal and/or to switch, based on the control signal, the device from a first active operating state to a second active operating state based on the control signal.

15. The method according to claim 14, wherein in the first active operating state, the liquid is delivered to the patient as a first liquid from the container as a first container and in the second active operating state a further liquid is delivered to the patient as a second liquid from a further container, and/or
wherein for switching the device from the first to the second active operating state, in addition to or instead of the pump as the first pump, a further pump is controlled as the second pump, and/or
wherein for switching the device from the first to the second active operating state, a multi-way valve is controlled.

16. The device according to claim 4, wherein the control device is configured to cause a pumping direction of the pump to be reversed and to determine, based on the measurement values acquired by the pressure measuring device, whether there is liquid on a side of the liquid-retaining filter membrane facing away from the pump.

17. The device according to claim 5, wherein the liquid-retaining filter membrane is arranged in an outlet region of the drip chamber.

18. The system according to claim 9, wherein the conduit comprises a drip chamber and wherein the liquid-retaining filter membrane is arranged in an outlet region of the drip chamber.

19. The system according to claim 13, wherein the multiport valve is a magnetic multiport valve and/or an actuator-operated multiport valve.
